(19) United States
(12) Patent Application Publication
(10) Pub. No.: US 2019/0175509 A1
(43) Pub. Date: Jun. 13, 2019

(54) High Drug Loaded Tablet Composition for Treating HIV

(71) Applicant: HETERO LABS LIMITED, Hyderabad, Telangana (IN)

(72) Inventors: Parthasarathi Reddy BANDI, Hyderabad, Telangana (IN); Khadgapathi PODILE, Hyderabad, Telangana (IN); Sunil Deviprasad TIWARI, Hyderabad, Telangana (IN); Prakash SHETIYA, Hyderabad, Telangana (IN); Balakrishnaiah MEDUM, Hyderabad, Telangana (IN)

(21) Appl. No.: 16/324,235

(22) PCT Filed: Jul. 28, 2017
(86) PCT No.: PCT/IB2017/054593

§ 371 Date: Feb. 8, 2019

(30) Foreign Application Priority Data
Aug. 8, 2016 (IN) IN201641026995

(51) Int. Cl. A61K 9/24; A61K 31/635; A61K 31/5365; A61K 31/426; A61P 31/18

(52) U.S. Cl. CPC A61K 9/209; A61K 31/635; A61P 31/18; A61K 31/426; A61K 31/5365

(57) ABSTRACT

The present invention relates to pharmaceutical antiretroviral compositions comprising a combination of antiretroviral agents, the manufacturing process thereof and use of the said compositions for the prevention, treatment or prophylaxis of diseases caused by retro viruses, specifically acquired immune deficiency syndrome or an HIV infection.

### PRIORITY

This patent application claims priority to Indian patent application number IN 201641026995, filed on Aug. 8, 2016, the contents of which are incorporated by reference herein in their entirety.

### FIELD OF THE INVENTION

The present invention relates to pharmaceutical antiretroviral compositions comprising a combination of antiretroviral agents, the manufacturing process thereof and use of the said compositions for the prevention, treatment or prophylaxis of diseases caused by retro viruses, specifically acquired immune deficiency syndrome or an HIV infection.

### BACKGROUND OF THE INVENTION

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus 111 (HTLV-111), now more commonly referred to as the human immunodeficiency viruses or HIV.

The treatment of HIV infection, known as cause of the acquired immunodeficiency syndrome (AIDS), remains a major medical challenge. HIV is able to evade immunological pressure, to adapt to a variety of cell types and growth conditions and to develop resistance against currently available drug therapies. Various available categories of drugs for treating HIV are nucleoside reverse transcriptase inhibitors (NRTIs), non-nucleoside reverse transcriptase inhibitors (NNRTIs), nucleotide reverse transcriptase inhibitors (NtRTIs), HIV-protease inhibitors, fusion inhibitors, CCR5 inhibitors and integrase inhibitors.

Although effective in suppressing HIV, each of these drugs, when used alone, is confronted with the emergence of resistant mutants. This led to the introduction of combination therapy of several anti-HIV agents usually having a different activity profile. In particular the introduction of “HAART” (Highly Active Anti-Retro viral Therapy) resulted in a remarkable improvement in anti-HIV therapy, leading to a large reduction in HIV-associated mortality. Even HAART may face the emergence of resistance, often due to non-adherence and non-persistence with antiretroviral therapy. In these cases HAART can be made effective again by replacing one of its components by one of another class. If applied correctly, treatment with HAART combinations can suppress the virus for many years, up to decades, to a level where it no longer can cause the outbreak of AIDS.

Combinations of anti-retrovirals create multiple obstacles to HIV replication to keep the number of offspring low and reduce the possibility of a superior mutation. If a mutation that conveys resistance to one of the drugs being taken arises, the other drugs continue to suppress reproduction of that mutation. Combination therapies greatly increase the ease with which they can be taken, which in turn increases the consistency with which medication is taken and thus their effectiveness over the long-term. Because of the complexity of selecting and following a regimen and the potential for side effects there lies an importance of taking medications regularly to prevent viral resistance.

The complex dosing regimens of HAART or other dosing regimens can be simplified by the application of combination dosage forms comprising two or more anti-HIV components. These could take the form of fixed dose combinations, e.g. compositions comprising predetermined doses of two or more anti-HIV agents. Most HIV inhibitors however need to be administered at relatively high doses so that often two or more doses need to be administered in order to reach the required therapeutic efficacy.

Currently available antiretroviral drugs for the treatment of HIV include nucleoside reverse transcriptase inhibitors (NRTI) or approved single pill combinations: Zidovudine or AZT (Retrovir®), didanosine or DDI (Videx®), stavudine or D4T (Zenith®), lamivudine or 3TC (Epivir®), zalcitabine or DDC (Hivid®), abacavir sulphate (Ziagen®), tenofovir disoproxil fumarate salt (Viread®), emtricitabine (Emtriva®), Combivir® (contains 3TC and AZT), Trizivir® (contains abacavir, 3TC and AZT); non-nucleoside reverse transcriptase inhibitors (NNRTI): nevirapine (Viramune®), delavirdine (Rescriptor®) and efavirenz (Sustiva®), peptidomimetic protease inhibitors or approved formulations: saquinavir (Invirase®, Fortovase®), indinavir (Crixivan®), ritonavir (Norvir®), nelfinavir (Viracept®), amprenavir (Agenerase®), atazanavir (Reyataz®), fosamprenavir (Lexiva®), Kaletra® (contains lopinavir and ritonavir), one fusion inhibitor enfuvirtide (T-20, Fuzeon®), Truvada® (contains Tenofovir disoproxil fumarate and Emtricitabine) and Atripla® (contains fixed-dose triple combination of tenofovir disoproxil fumarate, emtricitabine and efavirenz).

The current strategy recommended for the treatment of HIV infection is Highly Active Antiretroviral Therapy (HAART). HAART normally consists of a combination of two or more antiretroviral drugs (ARV) taken together in a single dosage form. Accordingly, inventors of the present invention have developed combination compositions with two or more antiretroviral drugs (ARV) taken together in a single dosage form.

Darunavir (TMC114) is a protease inhibitor approved in US and other countries, available under the trade name Prezista® in the form of darunavir ethanolate. It is chemically described as [(1S,2R)-3-[[(4-aminophenyl)sulfonyl](2-methylpropyl)amino]-2-hydroxy-1(phenylmethyl)propyl]-carbamic acid (3R,3aS,6aR)-hexahydrofuro[2,3-b]furan-3-yl ester monoethanolate.

Darunavir ethanolate is commercially available in US as 75 mg, 150 mg, 300 mg, 400 mg, 600 mg & 800 mg equivalent to base tablets and as equivalent to 100 mg base/ml oral suspension.

U.S. Pat. Nos. 5,843,946 & 7,700,645 discloses darunavir substance and its solvates including ethanolate.

PCT Publication No. 2009/013356 discloses darunavir tablet composition comprising 600 mg of darunavir as an active ingredient having a total weight of 1250 mg prepared by direct compression.

Dolutegravir sodium (DTG, GSK1349572) is an integrase inhibitor for the treatment of HIV infection. It is chemically described as (4R,12aS)-9-{[(2,4-difluorophenyl) methyl]carbamoyl}-4-methyl-6,8-dioxo-3,4,6,8,12,12a-hexahydro-2H-pyrido[1′,2′:4,5]pyrazino [2,1-b][1,3]oxazin-7-olate sodium.

Dolutegravir sodium is commercially available in US as 10 mg, 25 mg and 50 mg equivalent to base tablets under the brand name TIVICAY®.

U.S. Pat. No. 8,129,385 discloses dolutegravir substance.

Ritonavir is protease inhibitor for the treatment of HIV infection. It is chemically described as 10-Hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5R*,8R*,10R*,11R*)].

Ritonavir is marketed under the trade name Norvir® in the United States in the form of tablets, oral solution and capsules. It is commercially available in US as 100 mg tablets and as equivalent to 80 mg base/ml oral solution.

U.S. Pat. No. 5,541,206 discloses ritonavir substance.

Inventors of the present invention have developed a solid dosage form comprising novel combination of darunavir, dolutegravir and ritonavir (combination therapy) used for treating HIV, helps in preventing drug resistance. Also provides the dosage form with acceptable size, easy to swallow and reduced dosing frequency. Hence, present dosage form is beneficial in terms of pill burden and increase the patient compliance.

### SUMMARY OF THE INVENTION

The present invention relates to pharmaceutical unitary composition. In particular, the present invention relates to solid oral composition, comprising combination of anti-retro virals particularly, darunavir, dolutegravir and ritonavir and process of manufacturing the same.

The object of the present invention is to provide a pharmaceutical antiretroviral composition comprising darunavir, dolutegravir and ritonavir, in the form of a single unit dosage form.

One embodiment of the present invention relates to bilayered tablet composition comprising darunavir, dolutegravir and ritonavir and one or more pharmaceutically acceptable excipients.

Another embodiment of the present invention relates to a bilayered tablet composition comprising (i) darunavir or a pharmaceutically acceptable solvate thereof, (ii) dolutegravir or a pharmaceutically acceptable salt thereof and (iii) ritonavir and one or more pharmaceutically acceptable excipients.

Another embodiment of the present invention relates to a tablet composition comprising darunavir or a pharmaceutically acceptable solvate thereof and dolutegravir or a pharmaceutically acceptable salt thereof with one or more pharmaceutically acceptable excipients.

One another embodiment of the present invention relates to a bilayered tablet composition comprising: a) Darunavir or its pharmaceutically acceptable solvate thereof, b) Dolutegravir or its pharmaceutically acceptable salt thereof, c) Ritonavir, d) one or more pharmaceutically acceptable excipients, wherein darunavir and dolutegravir are in first compartment and ritonavir is in the second compartment, such that the total amount of darunavir, dolutegravir and ritonavir ranges from 40 to 65% by total weight of the composition.

By “total weight of the composition” is meant the weight of a tablet including the first and the second layers without coating.

One other embodiment of the present invention relates to pharmaceutical unitary tablet composition comprising a) 800 mg to 1200 mg of darunavir, b) 50 mg of dolutegravir, c) 100 mg of ritonavir and d) one or more pharmaceutically acceptable excipients.

One other embodiment of the present invention relates to a bilayered tablet composition comprising 800 mg of darunavir, 50 mg of dolutegravir and 100 mg ritonavir and one or more pharmaceutically acceptable excipients for once daily administration.

In yet another embodiment, the present invention is related to pharmaceutical unitary tablet composition comprising a) 400 mg of darunavir, b) 25 mg of dolutegravir, c) 50 mg of ritonavir and d) one or more pharmaceutically acceptable excipients.

Further embodiment of the present invention is relates to pharmaceutical unitary tablet composition comprising 400 mg of darunavir, 25 mg of dolutegravir and 50 mg ritonavir and one or more pharmaceutically acceptable excipients for twice daily administration.

In further embodiment, the present invention is relates to method of treating HIV or AIDS comprising administering a therapeutically effective amount of the unitary composition of the present invention.

### DETAILED DESCRIPTION OF THE INVENTION

The present invention relates to unitary composition comprising combination of anti-retro virals particularly, darunavir, dolutegravir and ritonavir and process of manufacturing the same.

One embodiment of the present invention relates to pharmaceutical unitary tablet comprising darunavir, dolutegravir and ritonavir with one or more pharmaceutically acceptable excipients thereof.

The term “active agent” as used herein according to the present invention refers to “darunavir”, “dolutegravir” and “ritonavir”.

The term “darunavir” as used herein according to the present invention includes darunavir in the form of free base or a pharmaceutically acceptable solvates or salts and its hydrates, preferably darunavir ethanolate.

The term “dolutegravir” as used herein according to the present invention includes dolutegravir in the form of free base or a pharmaceutically acceptable salt or solvate thereof. Preferably, dolutegravir sodium.

As used in this specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The term “excipient” means a pharmacologically inactive component such as a diluent, a binder, a disintegrant, a glidant, a lubricant, etc of a pharmaceutical product. The excipients that are useful in preparing a pharmaceutical composition are generally safe, non-toxic and are acceptable for human pharmaceutical use. Reference to an excipient includes both one and more than one such excipients.

The term “solid dosage form” or “dosage form” or “unitary composition” or “unitary tablet composition” or “bilayered tablet composition” or “unit dosage form” or “composition” or “formulation” as used herein refers to a solid dosage form suitable for oral administration, such as tablets including layered tablets, capsules, mini-tablets, spheroids, pellets, granules, pills and the like meant for oral administration.

By the term “core” is meant the granulate phase including the active agents and excipients.

By “total weight of the composition” is meant the weight of a tablet including the first and the second layers without coating.

The term “compartment” also used herein synonymously as “layer”.

The term “immediate release” as used herein refers to a dosage form that disintegrates and dissolves rapidly to release the actives.

Suitably, the pharmaceutical antiretroviral composition according to the present invention is presented in the solid dosage form suitable for oral administration. However, other dosage forms, such as liquid dosage forms and the like, may be envisaged under the ambit of the present invention.

Unit dosage form, according to the present invention, are preferably in the form of a tablet (disintegrating tablet, dissolving tablet, dispersible tablet, mouth dissolving tablets, tablet for oral suspension, immediate release tablets, extended release tablet, immediate and extended release tablets, matrix tablets), mini-tablet, granules, sprinkles (filled with powders, powders for reconstitution; beads; pellets; mini-tablets; film coated tablets; film coated tablets MUPS (multiple unit pellet system); orally disintegrating MUPS; pills; micro-pellets; small tablet units; MUPS; granules; effervescent granules; microspheres) or capsule (filled with powders, powder for reconstitution; beads; pellets; mini-tablets; film coated tablets; film coated tablets MUPS; orally disintegrating MUPS; pills; micro-pellets; small tablet units; MUPS; disintegrating tablets; dispersible tablets; granules; effervescent granules; microspheres), liquids such as suspension, emulsion, solution, syrup, elixir however, other dosage forms may also fall within the scope of this invention.

Preferably, the pharmaceutical antiretroviral composition, according to the present invention, is in the form of solid unit dosage forms including tablets and capsules, preferably in the form of immediate release tablets.

One embodiment of the present invention relates to pharmaceutical unitary tablet comprising darunavir, dolutegravir and ritonavir with one or more pharmaceutically acceptable excipients in the form of bilayered tablet, wherein, darunavir and dolutegravir is present in first compartment or layer and ritonavir is present in the second compartment or layer.

Another embodiment of the present invention relates to a tablet composition comprising darunavir or a pharmaceutically acceptable solvate thereof and dolutegravir or a pharmaceutically acceptable salt thereof with one or more pharmaceutically acceptable excipients.

Another embodiment of the present invention relates to pharmaceutical unitary tablet comprising darunavir, dolutegravir and ritonavir with one or more pharmaceutically acceptable excipients in the form of bilayered tablet, wherein, darunavir and dolutegravir is present in first compartment and ritonavir is present in the second compartment, wherein the weight of the darunavir, dolutegravir and ritonavir ranges from 40 to 65% by total weight of the composition, preferably 45 to 60%.

One other embodiment of the present invention is relates to pharmaceutical unitary tablet composition comprising a) 800 mg to 1200 mg of darunavir, b) 50 mg of dolutegravir, c) 100 mg of ritonavir and d) one or more pharmaceutically acceptable excipients.

One other embodiment of the present invention relates to a bilayered tablet composition comprising 800 mg of darunavir, 50 mg of dolutegravir and 100 mg ritonavir and one or more pharmaceutically acceptable excipients for once daily administration.

In yet another embodiment, the present invention is related to bilayered tablet composition comprising a) 400 mg of darunavir, b) 25 mg of dolutegravir, c) 50 mg of ritonavir and d) one or more pharmaceutically acceptable excipients.

Further embodiment of the present invention relates to bilayered tablet composition comprising 400 mg of darunavir, 25 mg of dolutegravir and 50 mg ritonavir and one or more pharmaceutically acceptable excipients for twice daily administration.

One or more pharmaceutically acceptable excipients of the present invention include diluents, binders, disintegrants, glidants, lubricants and the like.

Diluents include but are not limited to microcrystalline cellulose, powdered cellulose, lactose anhydrous, lactose monohydrate, dibasic calcium phosphate, tribasic calcium phosphate, starch, pregelatinized starch, calcium carbonate, calcium sulfate, magnesium carbonate, magnesium oxide, sucrose, dextrates, dextrin, dextrose, maltodextrin, mannitol, xylitol and sorbitol, and the like and combinations thereof.

Binders include but are not limited to hydroxypropyl cellulose, hydroxypropyl methylcellulose, polyvinyl pyrrolidone, pregelatinized starch, powdered acacia, gelatin, guar gum, carbomers and the like and combinations thereof.

Disintegrants include but are not limited to croscarmellose sodium, sodium starch glycolate, crospovidone, carboxymethyl cellulose calcium, starches such as corn starch, potato starch, pregelatinized starch and modified starches, clays, bentonite, microcrystalline cellulose and the like or combinations thereof.

Glidants include but are not limited to colloidal silicon dioxide, other forms of silicon dioxide, such as aggregated silicates and hydrated silica, magnesium silicate, magnesium trisilicate, talc, and the like and combinations thereof.

Lubricants include but are not limited to talc, magnesium stearate, calcium stearate, zinc stearate, stearic acid, palmitic acid, sodium stearyl fumarate, carnauba wax, hydrogenated vegetable oils, mineral oil, polyethylene glycols, and the like and combinations thereof.

The pharmaceutical antiretroviral composition, according to the present invention, may be prepared through various processes known in the art which includes, but are not limited to direct compression, wet granulation, dry granulation, melt granulation, melt extrusion, spray drying, solution evaporation or combinations thereof.

Pharmaceutical compositions of the present invention are prepared by either granulation techniques or direct compression. Preferably, first layer is prepared by granulation process and second layer is prepared by melt extrusion process.

Another aspect of the present invention relates to the bilayered tablet composition comprising darunavir, dolutegravir and ritonavir and one or more pharmaceutically acceptable excipients for once daily administration.

Even though such once-daily therapies represent a significant advantage, it would be highly desirable to faster the ease of administration, reduce the dosing frequency and help ensure patient compliance with such once daily therapies in the form of a unitary dosage form.

In yet another embodiment, the present invention relates to method of treating HIV or AIDS comprising administering a therapeutically effective amount of the unitary composition of the present invention.

Certain specific aspects and embodiments of this invention are described in further detail by the examples below, which are provided only for the purpose of illustration and are not intended to limit the scope of the invention in any manner.

### COMPARATIVE EXAMPLE

COMPARATIVE EXAMPLE
Darunavir, Dolutegravir and Ritonavir Monolithic tablet:

| Ingredients | mg/tab |
|---|---|
| Ritonavir | 100.00 |
| Copovidone | 458.810 |
| Colloidal silicon dioxide | 0.540 |
| Sorbitan monolaurate | 42.000 |
| Pre-lubrication | |
| Ritonavir extrudes | 601.350 |
| Darunavir Ethanolate | 867.296 |
| (Darunavir Ethanolate Eq to Darunavir) | |
| Dolutegravir sodium | 52.622 |
| (Dolutegravir sodium Eq to Dolutegravir) | |
| Mannitol | 75.082 |
| Sodium starch glycolate | 97.000 |
| Colloidal silicon dioxide | 0.750 |
| Lubrication | |
| Sodium stearyl fumarate | 11.000 |
| Core tablet weight | 1705.000 |

### Brief Manufacturing Process:

a) Sifting ritonavir, copovidone and colloidal silicon dioxide,
b) adding sorbitan monolaurate to step a) and passing the material through hot melt extruder and collecting the extrudes,
c) sifting darunavir ethanolate, dolutegravir sodium, mannitol, sodium starch glycolate and colloidal silicon dioxide,
d) mixing the extrudes of step b) with blend of step c),
e) Lubricating step d) with sodium stearyl fumarate and compressing into tablet, followed by coating.

### Dissolution Profile:

| | % of drug released | | |
|---|---|---|---|
| Time in minutes | Darunavir | Dolutegravir | Ritonavir |
| 30 | 23 | 25 | 28 |
| 60 | 35 | 35 | 36 |
| 90 | 41 | 41 | 41 |
| 120 | 46 | 46 | 46 |

### Example 1

### Darunavir, Dolutegravir and Ritonavir Bilayered Tablet:

| | |
|---|---|
| Ingredients | mg/tab |

DARUNAVIR + DOLUTEGRAVIR LAYER

| | |
|---|---|
| Darunavir Ethanolate | 867.296 |
| Dolutegravir sodium | 52.622 |
| Mannitol | 54.982 |
| Sodium starch glycolate | 32.000 |
| Povidone | 42.500 |
| Purified Water | Qs |
| Lubrication | |
| Sodium stearyl fumarate | 10.600 |
| Layer Weight | 1060.000 |

RITONAVIR LAYER

| | |
|---|---|
| Ritonavir | 100.000 |
| Copovidone | 458.810 |
| Colloidal silicon dioxide | 0.540 |
| Sorbitan monolaurate | 42.000 |
| Pre-lubrication | |
| Dibasic calcium phosphate anhydrous | 74.750 |
| Colloidal silicon dioxide | 0.750 |
| Lubrication | |
| Sodium stearyl fumarate | 6.150 |
| Layer Weight | 683.000 |
| Core Tablet weight | 1743.000 |
| Film coated tablet weight | 1795.29 |

### Brief Manufacturing Process:

### Preparation of Darunavir+Dolutegravir Layer:

a) Sifting darunavir, dolutegravir, mannitol and sodium starch glycolate and then loading into rapid mixer granulator and mixing for 10 minutes,
b) dissolving povidone in purified water and adding to the blend of step a) and kneading the wet mass, followed by drying, sifting to get the desired size granules,
c) sifting sodium stearyl fumarate,
d) mixing granules of step b) with sodium stearyl fumarate of step c) in blender

### Preparation of Ritonavir Layer:

a) Sifting ritonavir, copovidone and colloidal silicon dioxide and
b) adding sorbitan monolaurate to step a) and passing the material through hot melt extruder and collect the extrudes,
c) sifting dibasic calcium phosphate anhydrous and colloidal silicon dioxide and adding the material to step b) and blending for 10 min,
d) sifting sodium stearyl fumarate and adding to material of step c) and blending for 5 minutes;

### Compression of Bilayered Tablets:

Compressing the above blends of darunavir+dolutegravir in one layer and ritonavir in another layer into bilayered tablets, followed by film-coating.

### Dissolution Profile:

| | % of drug released | | |
|---|---|---|---|
| Time in minutes | Darunavir | Dolutegravir | Ritonavir |
| 30 | 63 | 82 | 59 |
| 60 | 91 | 89 | 90 |
| 90 | 97 | 89 | 96 |
| 120 | 99 | 89 | 98 |

### Example 2

### Darunavir, Dolutegravir and Ritonavir Bilayered Tablet:

| | |
|---|---|
| Ingredients | mg/tab |

DARUNAVIR + DOLUTEGRAVIR LAYER

| | |
|---|---|
| Darunavir Ethanolate | 867.296 |
| Dolutegravir sodium | 52.622 |
| Microcrystalline cellulose | 44.582 |
| Sodium starch glycolate | 45.000 |
| Colloidal silicon dioxide | 10.000 |
| Lubrication | |
| Sodium stearyl fumarate | 10.500 |
| Layer Weight | 1030.000 |

RITONAVIR LAYER

| | |
|---|---|
| Ritonavir | 100.000 |
| Copovidone | 349.750 |
| Colloidal silicon dioxide | 1.000 |
| Sorbitan monolaurate | 48.000 |
| Prelubrication | |
| Dibasic calcium phosphate anhydrous | 75.000 |
| Colloidal silicon dioxide | 0.750 |
| Lubrication | |
| Sodium stearyl fumarate | 5.500 |
| Layer Weight | 580.000 |
| Core Tablet weight | 1610.00 |
| Film coated tablet weight | 1658.300 |

### Brief Manufacturing Process:

### Preparation of Darunavir+Dolutegravir Layer:

a) Sifting darunavir, dolutegravir, microcrystalline cellulose, sodium starch glycolate and colloidal silicon dioxide and then loading into rapid mixer granulator and mixing for 10 minutes,
b) Compacting the step a), followed by milling, sifting to get the desired size granules,
c) sifting sodium stearyl fumarate,
d) mixing granules of step b) with sodium stearyl fumarate of step c) in blender

### Preparation of Ritonavir Layer:

a) Sifting ritonavir, copovidone and colloidal silicon dioxide and
b) adding sorbitan monolaurate to step a) and passing the material through hot melt extruder and collect the extrudes,
c) sifting dibasic calcium phosphate anhydrous and colloidal silicon dioxide and adding the material to step b) and blending for 10 min,
d) sifting sodium stearyl fumarate and adding to material of step c) and blending for 5 minutes;

### Compression of Bilayered Tablets:

Compress the above blends of darunavir+dolutegravir in one layer and ritonavir in another layer into bilayered tablets, followed by film-coating.

### Example 3

### Darunavir, Dolutegravir and Ritonavir Bilayered Tablet:

| | |
|---|---|
| Ingredients | mg/tab |

DARUNAVIR + DOLUTEGRAVIR LAYER

| | |
|---|---|
| Darunavir Ethanolate | 867.296 |
| Dolutegravir sodium | 52.622 |
| Microcrystalline cellulose | 35.00 |
| Sodium starch glycolate | 35.00 |
| Hydroxypropyl cellulose | 20.00 |
| Colloidal silicon dioxide | 10.00 |
| Purified Water | Qs |
| Lubrication | |
| Sodium stearyl fumarate | 10.50 |
| Layer Weight | 1030.418 |

RITONAVIR LAYER

| | |
|---|---|
| Ritonavir | 100.00 |
| Copovidone | 349.750 |
| Colloidal silicon dioxide | 1.00 |
| Sorbitan monolaurate | 48.00 |
| Prelubrication | |
| Dibasic calcium phosphate anhydrous | 15.00 |
| Colloidal silicon dioxide | 0.750 |
| Lubrication | |
| Sodium stearyl fumarate | 5.500 |
| Layer Weight | 520.00 |
| Core Tablet weight | 1550.418 |
| Film coated tablet weight | 1596.930 |

### Brief Manufacturing Process:

### Preparation of Darunavir+Dolutegravir Layer:

a) Sifting darunavir, dolutegravir, microcrystalline cellulose, sodium starch glycolate and colloidal silicon dioxide, then loading into rapid mixer granulator and mixing for 10 minutes, b) dissolving hydroxypropyl cellulose in purified water and adding to the blend of step a) and kneading the wet mass, followed by drying, sifting to get the desired size granules,
c) sifting sodium stearyl fumarate,
d) mixing granules of step b) with sodium stearyl fumarate of step c) in blender

### Preparation of Ritonavir Layer:

a) Sifting ritonavir, copovidone and colloidal silicon dioxide and
b) adding sorbitan monolaurate to step a) and passing the material through hot melt extruder and collect the extrudes,
c) sifting dibasic calcium phosphate anhydrous and colloidal silicon dioxide and adding the material to step b) and blending for 10 min,
d) sifting sodium stearyl fumarate and adding to material of step c) and blending for 5 minutes;

### Compression of Bilayered Tablets:

Compress the above blends of darunavir+dolutegravir in one layer and ritonavir in another layer into bilayered tablets, followed by film-coating.

TABLE A
Test Results (Dissolution profile):

| | % of drug released | | | | | |
|---|---|---|---|---|---|---|
| | Comparative Example | | | | | |
| Time | (Monolithic tablet) | | | Example 1 (Bi-layered tablet) | | |
| in minutes | Darunavir | Dolutegravir | Ritonavir | Darunavir | Dolutegravir | Ritonavir |
| 30 | 23 | 25 | 28 | 63 | 82 | 59 |
| 60 | 35 | 35 | 36 | 91 | 89 | 90 |
| 90 | 41 | 41 | 41 | 97 | 89 | 96 |
| 120 | 46 | 46 | 46 | 99 | 89 | 98 |

TABLE B
Innovator dissolution profiles for plain marketed tablets:

| | % of drug released | | |
|---|---|---|---|
| Time in | Darunavir | Dolutegravir | Ritonavir |
| minutes | (Prezista ®) | (Tivicay ®) | (Norvir ®) |
| 30 | 94 | 97 | 42 |
| 60 | 98 | 97 | 75 |
| 90 | 99 | 95 | 90 |
| 120 | 100 | 94 | 96 |

From the results shown in Table A and Table B, the bilayered tablet of the present invention could produce better dissolution without any delay as compared to plain marketed tablets of darunavir, dolutegravir and ritonavir.

Bilayered tablets (Example 1) drug release of three active agents found to be comparable with Innovator product drug release.

1. A unitary composition comprising:
a) darunavir or a pharmaceutically acceptable solvate thereof,
b) dolutegravir or a pharmaceutically acceptable salt thereof,
c) ritonavir, and
d) one or more pharmaceutically acceptable excipients;
wherein the darunavir and the dolutegravir are in a first compartment and the ritonavir is in a second compartment, and
wherein the total amount of darunavir, dolutegravir, and ritonavir combined ranges from 40 to 65% by weight of the total weight of the composition.

2. The unitary composition of claim 1, wherein the darunavir is present in an amount of 800 mg, the dolutegravir is present in an amount of 50 mg, and the ritonavir is present in an amount of 100 mg.

3. A unitary composition comprising 400 mg of darunavir, 25 mg of dolutegravir, 50 mg of ritonavir, and one or more pharmaceutically acceptable excipients, wherein the darunavir and the dolutegravir are in first compartment, and the ritonavir is in a second compartment.

4. The unitary composition of claim 1, wherein the first and second compartments are layers.

5. The unitary composition of claim 1, wherein the darunavir is darunavir ethanolate and the dolutegravir is dolutegravir sodium.

6. The unitary composition of claim 1, wherein amount of darunavir, dolutegravir and ritonavir ranges from 45 to 60% by total weight of the composition.

7. The unitary composition of claim 1, in the form of immediate release bilayered tablet, wherein a first layer comprises the first compartment, and a second layer comprises the second compartment.

8. The unitary composition of claim 1, wherein the first compartment is prepared by wet granulation, dry granulation, or direct compression.

9. The unitary composition of claim 1, wherein the second compartment is prepared by hot melt extrusion.

10. A method of treating HIV or AIDS comprising administering a therapeutically effective amount of the unitary composition of claim 1.

11. The unitary composition of claim 3, wherein the first and second compartments are layers.

12. The unitary composition according to claim 3, wherein the darunavir is darunavir ethanolate and the dolutegravir is dolutegravir sodium.

13. The unitary composition of claim 3, in the form of immediate release bilayered tablet, wherein a first layer comprises the first compartment, and a second layer comprises the second compartment.

14. The unitary composition of claim 3, wherein the first compartment is prepared by wet granulation, dry granulation, or direct compression.

15. The unitary composition of claim 3, wherein the second compartment is prepared by hot melt extrusion.

16. A method of treating HIV or AIDS comprising administering a therapeutically effective amount of the unitary composition of claim 3.
